(19) United States
(12) Patent Application Publication
(10) Pub. No.: US 2013/0109655 A1
(43) Pub. Date: May 2, 2013

(54) Method for mitigating of Prostaglandin E2 reducing side effects of non-steroidal anti-inflammatory drugs

(71) Applicants: Serghei Gargaun, Laval (CA); Ana Gargaun, Laval (CA)

(72) Inventors: Serghei Gargaun, Laval (CA); Ana Gargaun, Laval (CA)

(21) Appl. No.: 13/317,892

(22) Filed: Oct. 31, 2011

(51) Int. Cl. A61K 31/4439; A61K 31/444; A61K 31/4425; A61P 29/00; A61K 31/675; A61K 31/618; A61K 31/5415; A61K 31/455; A61K 31/4415

(52) U.S. Cl. 514/89; 514/343; 514/356; 514/355; 514/332; 514/277; 514/346; 514/351; 514/161; 514/226.5; 514/334

(57) ABSTRACT

This invention relates to a method of mitigating side effects of non-steroidal anti-inflammatory drugs, specifically to administration of a pyridine derivative to mitigate and/or prevent prostaglandin e2 reducing side effects of non-steroidal anti-inflammatory drugs. Also provided is the use of a pyridine derivative in the preparation of a medicament for the reduction of said side effects of non-steroidal anti-inflammatory drugs, and a pharmaceutical composition comprising a non-steroidal anti-inflammatory drug and a pyridine derivative.

### CROSS-REFERENCE TO RELATED APPLICATIONS

This application claims the benefit of Canadian patent application No. 2723561 filled 2010 Nov. 26 by the present inventors.

### FEDERALLY SPONSORED RESEARCH

Not Applicable

### SEQUENCE LISTING OR PROGRAM

Not Applicable

### BACKGROUND OF THE INVENTION

This application relates to a method for mitigating side effects of non-steroidal anti-inflammatory drugs, specifically to mitigation of prostaglandin e2 reducing side effects of non-steroidal anti-inflammatory drugs.

Prostaglandins are ubiquitously produced from arachidonic acid to elicit a diverse set of pharmacological effects mediating and modulating many physiological systems and have been implicated in a broad array of disorders. Prostaglandin e2 is a major prostaglandin in a number of physiological settings and plays key roles in various biological events.

Non-steroidal anti-inflammatory drugs, pharmacological agents that block prostaglandin biosynthesis are among the most frequently used prescription and over-the-counter drugs. In the United States, more than 70 million non-steroidal anti-inflammatory drug prescriptions are issued and 30 billion over-the-counter non-steroidal anti-inflammatory drug tablets are sold each year. They alleviate symptoms in acute and chronic inflammatory conditions induced by prostaglandins.

Non-steroidal anti-inflammatory drugs are generally well tolerated, but patients and clinicians continue to face the significant challenge of their serious complications. As yet, there are no safe and effective selective inhibitors of individual prostaglandin formation extensively used in medicine, which would enable further manipulation and diversion of the biosynthetic pathways. Clinically, there are available only non-steroidal anti-inflammatory drugs which reduce the levels of a large spectrum of prostaglandins. Although highly effective, the prostaglandin inhibitors of today's clinics are still rather blunt therapies at the molecular level, because they not only block the formation of individual pathogenic prostaglandins but also inhibit other “bystander” prostaglandins, like prostaglandin e2 (up to 50-85% (Mikkelsen, U. R. et al., 2008, J Appl Physiol, v 104, p: 534-7), (Vane, J. R., 1971, Nat New Biol, v 231, p: 232-5)) that are needed to maintain homeostasis.

The collateral prostaglandin e2 suppression leads to harmful iatrogenic side effects in systems and tissues in the range of prostaglandin e2 physiological action, as regulation of renal, uterine and other systems and organs. Reducing the prostaglandin e2 suppression side effect of non-steroidal anti-inflammatory drugs will considerably increase non-steroidal anti-inflammatory drugs safety and efficacy, especially when physiological functions of prostaglandin e2 are in addition abridged due to an additional suppression of physiological levels of prostaglandin e2 by concomitant medical conditions.

One of the main side effects of non-steroidal anti-inflammatory drugs is associated with suppression of prostaglandin e2 in kidneys. Prostaglandin e2 is by far the most predominant arachidonic acid metabolite produced in the kidney (Nasrallah, R. et al., 2007, Am J Physiol Renal Physiol, v 292, p: F278-84) and produces renal protection in vivo and in vitro (Lee, H. T., 2004, Am J Physiol Renal Physiol, v 287, p: F1111-2), (Paller, M. S. et al., 1992, Kidney Int, v 42, p: 1345-54).

Renal side effects of non-steroidal anti-inflammatory drugs are rare, sometimes transient and often reversible upon drug withdrawal. However, due to non-steroidal anti-inflammatory drugs use by a great many patients, the small percentage translates to a serious problem involving many patients particularly in high risk subjects. For instance, approximately 50 million Americans per year are likely to take NSAIDs, and some 500,000 (1%) of them are thought to experience renal side effects (Heptinstall, R. H., 2007, Hepinstall's Pathology of the Kidney, v 2, p: 1098, Lippincott Williams & Wilkins), (Whelton, A., 1999, Am J Med, v 106, p: 13S-24S). The incident rate and the severity of the renal side effect increases in patients with diabetes, heart failure, renal dysfunction and in the elderly (Harirforoosh, S. et al., 2009, Expert Opin Drug Saf, v 8, p: 669-81). Inhibition of renal prostaglandin synthesis with non-steroidal anti-inflammatory drugs reduces renal venous and urinary concentrations of prostaglandin e2 by 50 to 75% (Zambraski, E. J. et al., 1979, Am J Physiol, v 236, p: F552-8). By blocking prostaglandin e2, non-steroidal anti-inflammatory drugs cause an increase in water absorption, reduction in urine volume, and fluid retention (Zusman, R. M., 1981, Med Clin North Am, v 65, p: 915-25). Prostaglandin e2 inhibition results in sodium retention, which leads to hypertension, peripheral edema (Francois, H. et al., 2007, J Am Soc Nephrol, v 18, p: 1466-75) and potentially exacerbation of heart failure (Sanghi, S. et al., 2006, Cardiovasc Hematol Disord Drug Targets, v 6, p: 85-100).

The prostaglandins have powerful smooth muscle- and vascular-modulating properties, which can induce as well as alleviate various disorders. In pregnant uterus production of prostaglandin e2 induces labor and initiates uterine contractions (Rama Sastry, B. V. et al., 1999, Pharmacology, v 58, p: 70-86). In non-pregnant uterus it was found that prostaglandin e2 produces myometrial relaxation and is a vasodilator at the time of menstruation, while prostaglandin F2a has vasoconstrictor and smooth muscle-stimulating properties with a maximal effect on the premenstrual myometrium (Dawood, M. Y. et al., 2007, Prostaglandins Other Lipid Mediat, v 83, p: 146-53), (Salamonsen, L. A. et al., 1999, Baillieres Best Pract Res Clin Obstet Gynaecol, v 13, p: 161-79), (Abraham, G. E., 1978, Clin Obstet Gynecol, v 21, p: 139-45), (Lundstrom, V., 1977, Acta Obstet Gynecol Scand, v 56, p: 167-72).

Primary dysmenorrhea is estimated to be present in 40-50% of young women (Ylikorkala, O. et al., 1978, Am J Obstet Gynecol, v 130, p: 833-47). The main reason for the abnormally enhanced contractility pattern seen in dysmenorrheic women during menstruation is an increased PGF2a/PGe2 ratio (Bygdeman, M. et al., 1979, Acta Obstet Gynecol Scand Suppl, v 87, p: 33-8) largely due to prostaglandin F2a increased levels (Chan, W. Y. et al., 1978, Prostaglandins, v 15, p: 365-75), (Pulkkinen, M. O. et al., 1978, Prostaglandins, v 15, p: 543-50), (Zahradnik, H. P. et al., 1978, Dtsch Med Wochenschr, v 103, p: 1270-3), (Pickles, V. R. et al., 1965, J Obstet Gynaecol Br Commonw, v 72, p: 185-92). The resultant uterine muscle ischemia and hypoxia are believed to be the origin of pain in primary dysmenorrhea (Iacovides, S. et al., 2009, Sleep, v 32, p: 1019-26), (Dawood, M. Y., 2006, Obstet Gynecol, v 108, p: 428-41), (Harel, Z., 2004, J Pediatr Adolesc Gynecol, v 17, p: 75-9), (Dawood, M. Y., 1985, J Reprod Med, v 30, p: 154-67).

Given the involvement of prostaglandins in the etiology of primary dysmenorrhea, the current most accepted approach for its treatment is to inhibit the synthesis of the uterine contractor prostaglandins. Non-steroidal anti-inflammatory drugs reduce levels of endometrial prostaglandins and represent the most common pharmacological approach for treatment of primary dysmenorrhea (Dawood, M. Y., 2006, Obstet Gynecol, v 108, p: 428-41). The accumulated data clearly explain the analgesic effect of non-steroidal anti-inflammatory drugs correlated with the reduction of basal tonus and with, presumably, also decreased vasoconstriction and improved circulation within the myometrium (Lundstrom, V. et al., 1979, Acta Obstet Gynecol Scand Suppl, v 87, p: 51-6).

Despite this success there is a serious drawback to the use of non-steroidal anti-inflammatory drugs in primary dysmenorrhea related to the responder rate that has been observed for these drugs and that has not yet been resolved. In general it is found that about only 80-85% of the patients treated with non-steroidal anti-inflammatory drugs respond well to such treatment (Dawood, M. Y., 2006, Obstet Gynecol, v 108, p: 428-41). Nevertheless, ten to fifteen percent of women with dysmenorrhea still suffer from dysmenorrhea symptoms which limits daily activity and is unimproved by analgesics (Sundell, G. et al., 1990, Br J Obstet Gynaecol, v 97, p: 588-94), (Andersch, B. et al., 1982, Am J Obstet Gynecol, v 144, p: 655-60).

After administration of non-steroidal anti-inflammatory drugs, with the inhibition of muscle stimulating and vasoconstrictor prostaglandins, specifically prostaglandin F2a, the non-steroidal anti-inflammatory drugs process inhibition of prostaglandin e2, which as a potent myorelaxant can inhibit uterine contractility even in dysmenorrheic patients (Bygdeman, M. et al., 1979, Acta Obstet Gynecol Scand Suppl, v 87, p: 33-8). Suppression of prostaglandin e2 trades off the beneficial physiological myorelaxant and vasodilator effects of prostaglandin e2 on dysmenorrheic ischemia and could be a cause that not all patients are relieved from the dysmenorrhea symptoms.

The undesirable suppression of physiological levels of prostaglandin e2 in dysmenorrhea by non-steroidal anti-inflammatory drugs increases the already elevated, due to high prostaglandin F2a levels, PGF2a/PGe2 ratio. The appropriate ratio of PGF2a/PGe2 is necessary for the regulation of the myometrial tone (Creatsas, G. et al., 1990, Eur J Obstet Gynecol Reprod Biol, v 36, p: 292-8), (Gantt, P. A. et al., 1981, Pediatr Clin North Am, v 28, p: 389-95), (Pickles, V. R. et al., 1965, J Obstet Gynaecol Br Commonw, v 72, p: 185-92), (Pickles, V. R., 1957, Nature, v 180, p: 1198-9), therefore, all pharmacological measures that are capable to increase the collaterally suppressed levels of the myorelaxant and vasodilator prostaglandin e2 by non-steroidal anti-inflammatory drugs, normalizing the PGF2a/PGe2 ratio will be able to treat dysmenorrheic pain.

### BRIEF SUMMARY OF THE INVENTION

The invention provides methods and compositions for mitigating said side effects of non-steroidal anti-inflammatory drugs that can be delivered to a patient, either concurrently with, or before or after non-steroidal anti-inflammatory drug therapy.

In one embodiment of the present application is provided a method for mitigating side effects of non-steroidal anti-inflammatory drugs in a patient being treated with a non-steroidal anti-inflammatory drug, specifically mitigation of prostaglandin e2 reducing side effects of non-steroidal anti-inflammatory drugs, comprising administration of a pyridine derivative.

In one aspect, the administration is concurrent with the non-steroidal anti-inflammatory drugs treatment.

In a further aspect, the administration is previous to the non-steroidal anti-inflammatory drugs treatment.

In a further aspect, the administration is after the non-steroidal anti-inflammatory drugs treatment.

In another embodiment of the present application are provided pharmaceutical compositions comprising non-steroidal anti-inflammatory drugs and pyridine derivatives.

In another embodiment is provided use of pyridine derivatives in preparation of a medicament for reduction of said side effects of non-steroidal anti-inflammatory drugs.

### BRIEF DESCRIPTION OF DRAWINGS

In the drawings, which form a part of this specification,

FIG. 1. is the inventors' compilation of graphs A, D and E from FIG. 4. from publication “The immunosuppressive effects of nicotine during human mixed lymphocyte reaction” (Takahashi, H. K. et al., 2007, Eur J Pharmacol, v 559, p: 69-74), from which inventors using UN-SCAN-IT® Graph Digitizing System from Silk Scientific Corporation (Utah 84059 USA) extracted three concentration coordinates of prostaglandin e2 (337 pM, 768 pM and 761 pM) and on which marked said coordinates on the bottom of said graphs. Only data in the absence (open circles) of IL-18 were used;

FIG. 2. is the inventors' compilation of graphs B, E and F from FIG. 3. from publication “Effect of nicotine on IL-18-initiated immune response in human monocytes” (Takahashi, H. K. et al., 2006, J Leukoc Biol, v 80, p: 1388-94), from which inventors using UN-SCAN-IT® extracted three concentration coordinates of prostaglandin e2 (0.35 nM, 0.5 nM and 0.55 nM) and on which marked said coordinates on the bottom of said graphs. Only data in the absence (open circles) of IL-18 were used;

FIG. 3. is prostaglandin e2 section extracted by inventors from graph of FIG. 1. from publication “Increased compliance of niceritrol treatment by addition of Aspirin®: relationship between changes in prostaglandins and skin flushing.” (Nozaki, S. et al., 1987, Int J Clin Pharmacol Ther Toxicol, v 25, p: 643-7). Inventors marked on said figure extracted by inventors by using UN-SCAN-IT® basal (52.3, 33.6, 26.6, 21.2, 19.1, 18.1, 16.8, 16.0, 15.1, 13.9), and after niceritrol and Aspirin® administration (46.2, 16.6, 42.0, 44.1, 8.2, 11.4, 18.6, 9.9, 27.6, 6.2) concentration coordinates (pg/ml) of prostaglandin e2.

FIG. 4. is graph from FIG. 3. from publication “Prostaglandins contribute to the vasodilation induced by nicotinic acid” (Eklund, B. et al., 1979, Prostaglandins, v 17, p: 821-30), from which inventors using UN-SCAN-IT® extracted digital coordinates of actual basal release (35.6±29.8) of radioimmunoassayed prostaglandin E (R-PGE) and of actual release (pg/100 ml tissue×min) of R-PGE after treatment with naproxen and nicotinic acid (8.2±13.8, 10.6±16.6, 18.8±16.2 and 15.1±13), and on which marked said coordinates on the bottom of said graph. Only data in the presence (open circles) of naproxen were used.

FIG. 5. is a table which depicts the inventors' demonstration of effect of treatment of isolated rat hearts with indomethacin and pyridine derivatives on prostaglandin e2 like substance outflow vs. control group, based on inventors' assessment of data from “Effects of pyridine and some of its derivatives on prostaglandin synthesis” publication (Sahin, I. et al., 1984, Arch Int Pharmacodyn Ther, v 270, p: 324-9). t-test for means of two groups (Student's t-test) statistical analysis calculated by inventors shows that the differences of means of outflow after treatment with indomethacin and pyridine derivatives vs. control group are statistically non-significant.

### DETAILED DESCRIPTION OF THE INVENTION

The present inventors have surprisingly found that the aforementioned side effects of non-steroidal anti-inflammatory drugs can be mitigated by providing a method and compositions for reducing and/or preventing prostaglandin e2 deficiency in non-steroidal anti-inflammatory treatment in mammalian patients that comprises administration of an effective amount of a pyridine derivative. The present application is based upon the inventors' discovery that use of a pyridine derivative can mitigate the suppressing side effect of non-steroidal anti-inflammatory drugs on prostaglandin e2 in mammals. It has never been previously conjectured, investigated or proposed the possibility of using a pyridine derivative that will selectively mitigate the absolute or relative deficiency of prostaglandin e2 in non-steroidal anti-inflammatory treatment.

The present application is based on experimental data extracted by inventors from previously published peer-review articles, additional calculations to the data, anew assessment and reconsideration of the new obtained data as performed by the inventors. If a graph for the average concentration population data was available in the original reference, the experimental data points were obtained by using the UN-SCAN-IT® Graph Digitizing System to read the data from the published figures. This digitizing system allows one to convert graphs from pdf-files to digital concentration data. (May, R. A. et al., 2008, Journal of the American Chemical Society, v 130, p: 7516, American Chemical Society), (Edwards, P. M., 2002, Journal of Chemical Information and Computer Sciences, v 42, p: 1272, American Chemical Society), (Silbert, M. D., 2000, Canadian Chemical News, v 52, p: 8), (Carter, P. J. et al., 1997, Journal of the American Chemical Society, v 119, p: 11135, American Chemical Society).

The nicotine (a pyridine derivative), non-steroidal anti-inflammatory drugs and prostaglandin e2 data were from two publications by Takahashi et al. (Takahashi, H. K. et al., 2007, Eur J Pharmacol, v 559, p: 69-74), (Takahashi, H. K. et al., 2006, J Leukoc Biol, v 80, p: 1388-94). The objective of the Takahashi et al. (2007) study was examination of the effect of nicotine on immune response in the presence or absence of IL-18 and the involvement of nicotinic acetylcholine receptor a7 subunit in the actions of nicotine. To investigate the mechanism of nicotine action, the effect of COX-2 and protein kinase A inhibitors on the nicotine-treated mixed lymphocyte reaction was determined. Inventors investigated only the nicotine, selective and non-selective non-steroidal anti-inflammatory drugs and prostaglandin e2 data in the absence of IL-18.

Normal human peripheral blood cells treated with 100 mcM nicotine were incubated with increasing concentrations ranging from 0.1 to 100 mcM of a non-selective COX-2 inhibitor, indomethacin, a non-selective non-steroidal anti-inflammatory drug, and a selective COX-2 inhibitor, NS398, a selective non-steroidal anti-inflammatory drug (John-Aryankalayil, M. et al., 2009, Molecular cancer therapeutics, v 8, p: 261-73), (Pan, M. R. et al., 2008, Exp Biol Med (Maywood), v 233, p: 456-62). In the description of the study results, authors of the publication mentioned that indomethacin and NS398 again suppressed nicotine-initiated prostaglandin e2 production.

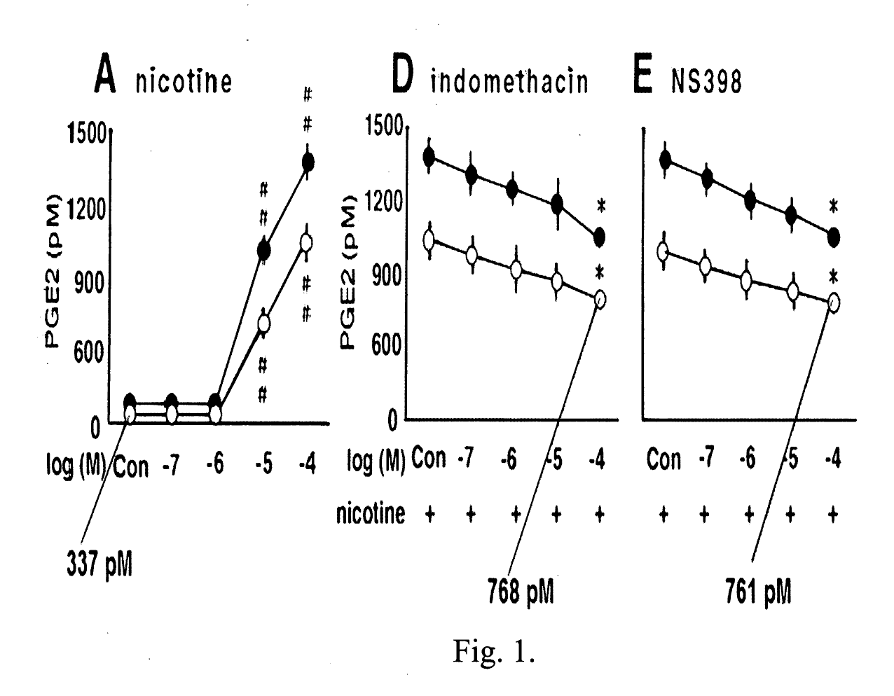

Inventors analysed graphs from the FIG. 1. and noticed that during treatment of the nicotine pre-treated cells with all concentrations of non-steroidal anti-inflammatory drugs (graphs D and E) the prostaglandin e2 concentrations were not supressed below the basal levels (graph A; Con, 0 M). Inventors used UN-SCAN-IT® to obtain digital concentration data of prostaglandin e2 at treatment with the highest concentration (log M=−4) of indomethacin and NS398, and noticed that prostaglandin e2 concentrations of the nicotine pre-treated cells were 768 pM (graph D) and 761 pM (graph E) respectively, while basal (NSAIDs and nicotine non-treated) concentration of prostaglandin e2 was 337 pM (graph A; Con, 0 M). Inventors concluded that after treatment even with the highest concentration of indomethacin and NS398, prostaglandin e2 concentrations (768 pM and 761 pM, respectively) of the nicotine pre-treated cells were not reduced below the basal concentration (337 pM) of prostaglandin e2. Thus, inventors have found that in non-steroidal anti-inflammatory drug treatment, use of a pyridine derivative does not allow non-steroidal anti-inflammatory drugs to suppress prostaglandin e2 below the basal levels.

In the second Takahashi et al. publication (2006) the authors examined the effects of nicotine on the expression of ICAM-1, B7.2, and CD40 and production of IL-12, IFN-g, and TNF-a in IL-18-treated peripheral blood mononuclear cells and monocytes, and also investigated the involvement of prostaglandin e2 production in mediating these effects. Inventors investigated only the nicotine, selective and non-selective non-steroidal anti-inflammatory drugs and prostaglandin e2 data in the absence of IL-18. In the description of the study results, authors of the publication mentioned that a nonselective or a selective COX-2 inhibitor, indomethacin or NS398, inhibited the nicotine-induced PGE2 production.

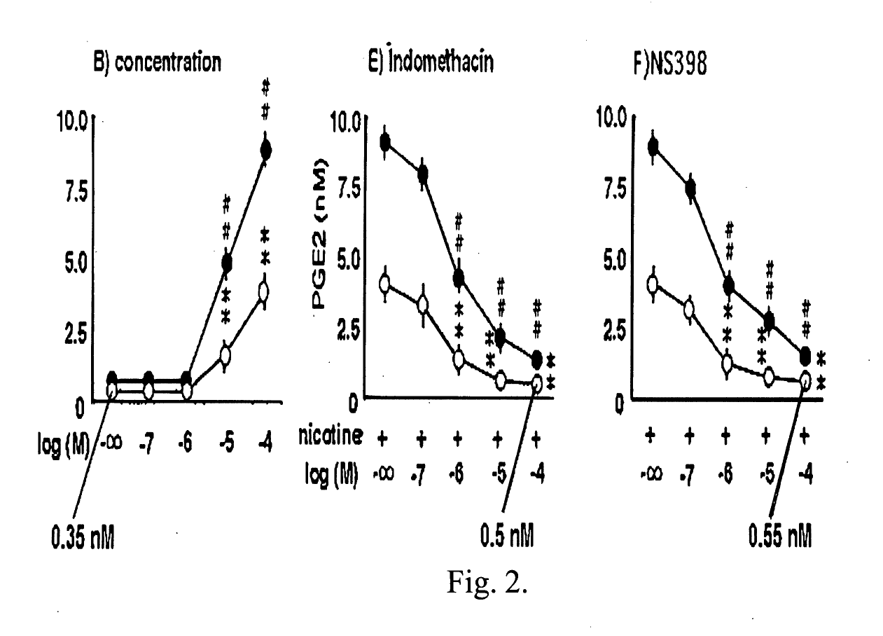

Inventors analysed graphs from the FIG. 2. and noticed that during treatment of the nicotine pre-treated cells with all concentrations of non-steroidal anti-inflammatory drugs (graphs E and F) the prostaglandin e2 concentrations were not supressed below the basal levels (graph B). Inventors used UN-SCAN-IT® to obtain digital concentration data of prostaglandin e2 at the treatment with the highest concentration (log M=−4) of indomethacin and NS398, and identified that prostaglandin e2 concentrations of the nicotine pre-treated cells were 0.50 nM (graph E) and 0.55 nM (graph F), respectively, while basal concentration of prostaglandin e2 was 0.35 nM (graph B). Inventors noticed the same effect found by inventors in the previously analysed Takahashi et. al. (2007) study, that after treatment even with the highest concentration of indomethacin and NS398, prostaglandin e2 concentrations of the nicotine pre-treated cells were not reduced below the basal concentration of prostaglandin e2.

The inventors' findings from the Takahashi et al. (2006) publication confirms the inventors' conclusion of the investigation of the Takahashi et al. (2007) publication that in non-steroidal anti-inflammatory drug treatment use of a pyridine derivative does not allow non-steroidal anti-inflammatory drugs to reduce prostaglandin e2 concentration below the basal concentration. The authors mentioned in both publications that indomethacin or NS398 inhibited the nicotine-induced prostaglandin e2 production, but did not notice and did not describe in the publications the revealed by inventors new findings.

The niceritrol (a pyridine derivative), Aspirin® (a non-steroidal anti-inflammatory drug) and prostaglandin e2 data were from the publication “Increased compliance of niceritrol treatment by addition of Aspirin®: relationship between changes in prostaglandins and skin flushing” (Nozaki, S. et al., 1987, Int J Clin Pharmacol Ther Toxicol, v 25, p: 643-7).

The objective of the Nozaki, S. et al. study was investigation of incidence of flushing by niceritrol intake and possibility to prevent it with Aspirin® in 10 normal male volunteers in relation to changes in serum levels of prostaglandin e2 and 6 keto-PGF1a after niceritrol and Aspirin® intake. Inventors investigated only the niceritrol, Aspirin® and prostaglandin e2 data.

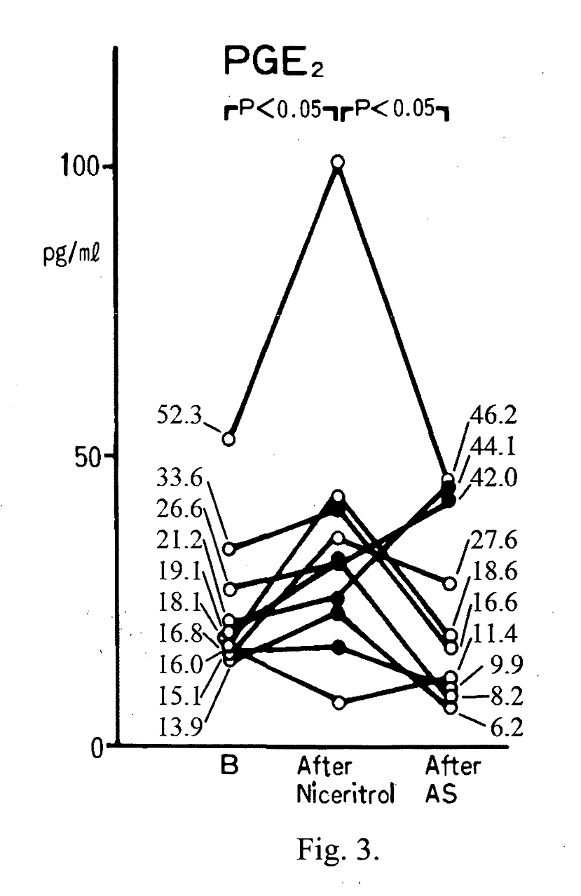

Inventors used UN-SCAN-IT® to obtain digital concentration data from prostaglandin e2 section of the graph from the FIG. 1. of the publication (FIG. 3.). The obtained by inventors serum basal (52.3, 33.6, 26.6, 21.2, 19.1, 18.1, 16.8, 16.0, 15.1, 13.9), and after niceritrol and Aspirin® administration (46.2, 16.6, 42.0, 44.1, 8.2, 11.4, 18.6, 9.9, 27.6, 6.2) prostaglandin e2 digital levels (pg/ml) were statistically analysed using paired t-test. Inventors found that there was not statistically significant (P=0.964) difference between the means (M±SD) of the basal prostaglandin e2 concentration (23.3±11.8 pg/ml) and after the administration of niceritrol and Aspirin® (23.1±15.8 pg/ml).

After the statistical analysis of the obtained digital data, inventors concluded that administration of Aspirin® could not reduce below basal concentrations the elevated by niceritrol prostaglandin e2 levels. Authors of the publication mentioned that the administration of niceritrol significantly increased serum levels of prostaglandin e2 and the addition of Aspirin® significantly decreased the levels, but did not notice and not describe in the publication the new findings revealed by the inventors' investigation. Therefore, inventors' findings from the Nozaki, S. et al. study have confirmed the inventors' conclusions from the investigation of the Takahashi et al. publications that administration of a pyridine derivative does not allow reduction of prostaglandin e2 concentration below the basal levels by a non-steroidal anti-inflammatory drug.

The nicotinic acid (a pyridine derivative), naproxen (a non-steroidal anti-inflammatory drug) and radioimmunoassayed prostaglandin E (R-PGE) data were from the “Prostaglandins contribute to the vasodilation induced by nicotinic acid” study publication (Eklund, B. et al., 1979, Prostaglandins, v 17, p: 821-30), where the publication authors investigated the hypothesis that nicotinic acid elicits its vasodilating effect by a mechanism that involves stimulation of the endogenous prostaglandin synthesis. The authors measurements showed that the basal R-PGE concentration in the forearm venous effluent from healthy male volunteers was 21±10 pg/ml. Infusion of nicotinic acid resulted in a significant increase in the plasma concentration of R-PGE to 49±7 pg/ml, which was reduced after naproxen treatment to 8±2 pg/ml. According to the publication authors' justifications, the increase in R-PGE after infusion of nicotinic acid was probably due to mainly prostaglandin e2.

Inventors performed an investigation of the data provided in the publication. As calculated by the inventors, the statistical significance (Student's t-test) of the difference of the mean (M±SE) basal R-PGE concentration (21±10 pg/ml, n=6) in the forearm venous effluent vs. the mean plasma R-PGE concentration (8±2 pg/ml, n=3) after naproxen treatment and nicotinic acid infusion was found non-significant (P=0.41). So, based on the statistical analysis performed by the inventors, the inventors have found that after naproxen treatment of nicotinic acid exposed tissues, plasma level of R-PGE was not suppressed below basal levels, probably due to the increased by nicotinic acid mainly prostaglandin e2.

Thus, accordingly to these new statistical data obtained by inventors from the publication, inventors have found that tissue exposure to a pyridine derivative does not allow reduction of prostaglandin E concentration below the basal levels by a non-steroidal anti-inflammatory drug (probably due to the increased by nicotinic acid mainly prostaglandin e2). The publication's authors mentioned that the increase in plasma R-PGE following nicotinic acid was totally abolished after naproxen, and the plasma R-PGE remained low during the entire nicotinic acid infusion, but did not notice and did not describe in the publication the revealed by inventors new findings

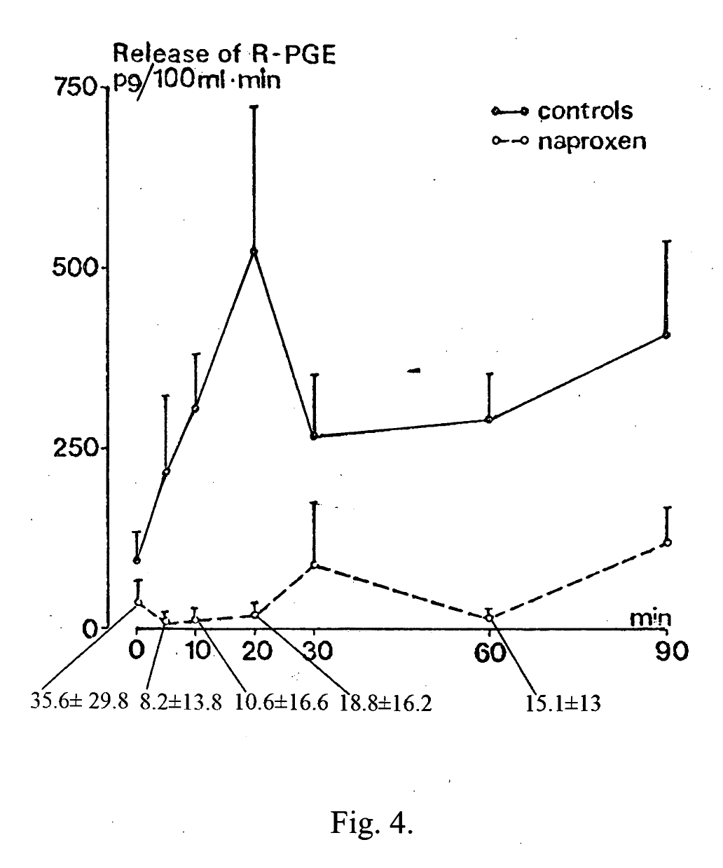

Furthermore, inventors performed an investigation of actual release of R-PGE (estimated from the product of the plasma concentration of R-PGE×the forearm blood flow) from the same publication. After visual investigation of the graph (FIG. 4.) inventors observed that at two time points (30, 90 min) the graph line (open circles) of the release of R-PGE after treatment with naproxen and nicotinic acid was above the basal release of R-PGE (0 min) while at four time was points (5, 10, 20 and 60 min) below the levels of the basal release of R-PGE.

Inventors decided to obtain digital data of the release of R-PGE after treatment with naproxen and nicotinic acid at the points below the levels of the basal release of R-PGE and digital data of basal release of R-PGE from the graph by using UN-SCAN-IT®. To compare the differences of the obtained digital data (8.2±13.8, 10.6±16.6, 18.8±16.2 and 15.1±13 pg/100 ml tissue×min, respectively) of release of R-PGE at the points below the levels of the basal release of R-PGE vs. digital data (35.6±29.8 pg/100 ml tissue×min) of basal release of R-PGE, inventors completed t-test for means (M±SE) of two groups (Student's t-test, n=3) and found the differences statistically non-significant (P=0.45, 0.5, 0.65 and 0.56, respectively). So, accordingly to the inventors' visual investigation of the graph and the statistical analysis of the digital data obtained from the graph, the release of R-PGE after treatment with naproxen and nicotinic acid was not significantly reduced below the basal release of R-PGE at any point during the measured release of R-PGE.

The publication's authors mentioned that naproxen completely blocks the increase in forearm release of R-PGE induced by nicotinic acid, but did not notice and did not describe in the publication the new findings revealed by the inventors' investigation. Inventors' examination of the data on actual release of R-PGE confirmed the inventors' findings from the data on plasma concentration of R-PGE from the same publication that exposure to a pyridine derivative does not allow non-steroidal anti-inflammatory drugs to decrease the prostaglandin E levels below basal levels, and as a result reduces the prostaglandin E suppression side-effect (probably due to the increased by nicotinic acid mainly prostaglandin e2) of non-steroidal anti-inflammatory drugs.

The data on several pyridine derivatives, indomethacin (a non-steroidal anti-inflammatory drug) and prostaglandin e2 like substance (PGe2-LS) were from “Effects of pyridine and some of its derivatives on prostaglandin synthesis” publication (Sahin, I. et al., 1984, Arch Int Pharmacodyn Ther, v 270, p: 324-9). The authors of the publication investigated the effects of pyridine and some of its derivatives on the release of a PGe2-LS from isolated perfused rat hearts (n=5). At 1×10⁻³ M concentration all pyridine derivatives studied increased the release of PGe2-LS significantly. Indomethacin treatment reduced PGe2-LS release stimulated by pyridine derivatives.

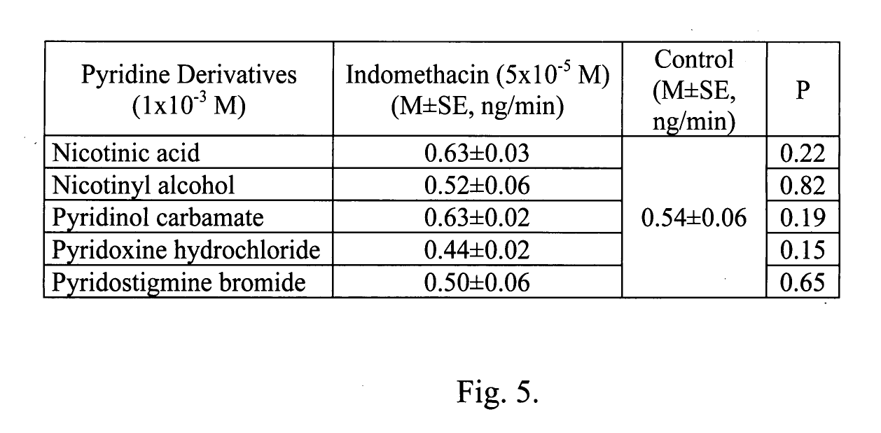

Inventors calculated the statistical significance of differences of means (M±SE) of PGe2-LS release (ng/min) in samples treated with indomethacin and pyridine derivatives (nicotinic acid=0.63±0.03, nicotinyl alcohol=0.52±0.06, pyridinol carbamate=0.63±0.02, pyridoxine hydrochloride=0.44±0.02 and pyridostigmine bromide=0.50±0.06) vs. mean (0.54±0.06) of control group samples. The completed by inventors t-test for means of two groups (Student's t-test) showed that the differences were statistically non-significant for all pyridine derivatives (FIG. 5.).

These new data obtained by the inventors' statistical analysis have revealed that in indomethacin treatment of isolated rat hearts, exposure to pyridine derivatives does not decrease PGe2-LS release significantly compared to the control group. The publication authors noticed that indomethacin treatment reduced PGe2-LS release stimulated by pyridine derivatives, but did not observe and did not mention that PGe2-LS stimulated release by a pyridine derivative is reduced by a non-steroidal anti-inflammatory drug to the levels of the control group, but not below. So, inventors have found that tissue treatment with a pyridine derivative can prevent decrease of PGe2-LS release below the range of basal levels in non-steroidal anti-inflammatory drugs treatment.

The new data obtained by inventors from the investigation of the previously published peer reviewed publications have shown for the first time that use of pyridine derivatives in non-steroidal anti-inflammatory treatment does not allow suppression of prostaglandin e2 levels by non-steroidal anti-inflammatory drugs below the basal (physiological) levels. The authors of the investigated publications confirmed the known effect of suppression of prostaglandin e2 by non-steroidal anti-inflammatory drugs, but did not observe and respectively did not mention that in the presence of a pyridine derivative, extent of suppression of prostaglandin e2 levels by non-steroidal anti-inflammatory drugs was significantly mitigated and the levels were not reduced below the physiological limits of prostaglandin e2. As well, the authors of the investigated publications observed that the pyridine derivatives increased the levels of prostaglandin e2, but did not observe and did not mention that the increasing of PGE2 by pyridine derivatives does not allow suppression of PGE2 by NSAIDs below the physiological levels.

Due to publication in peer-reviewed journals, the facts observed and mentioned in the investigated by inventors studies that NSAIDs inhibited the increased by pyridine derivatives prostaglandin e2 levels have become common general knowledge to any person of skills in the art, and as a result, the extent of suppression by non-steroidal anti-inflammatory drugs of elevated by pyridine derivatives prostaglandin e2 concerning prostaglandin e2 basal (physiological) levels has never been investigated. Therefore, it has not previously been considered the possibility of using of pyridine derivatives in non-steroidal anti-inflammatory treatment, specifically to mitigate the prostaglandin e2 reducing side effects of non-steroidal anti-inflammatory drugs. Thus, the effect of mitigation by pyridine derivatives of suppression of prostaglandin e2 in non-steroidal anti-inflammatory treatment found by inventors was an unexpected new result.

Although the use of non-steroidal anti-inflammatory drugs and pyridine derivatives may have been described in the prior art, it has not been observed before that pyridine derivatives addition do not allow non-steroidal anti-inflammatory drugs to decrease prostaglandin e2 levels below the physiological levels, as in use of non-steroidal anti-inflammatory drugs without administration of pyridine derivatives.

A great number of studies on prostaglandin mediated disorders indicate that ingestion of non-steroidal anti-inflammatory drugs decreases considerably not only levels of prostaglandins mediating pathological processes, but also physiological levels of prostaglandin e2, reducing its beneficial physiological effects, which is especially important in some medical conditions such as disorders of the kidneys, diabetes, dysmenorrhea, but not limited to. As a result, suppression of physiological levels of prostaglandin e2 by non-steroidal anti-inflammatory drugs affects the safety and trades off the therapeutic effect of non-steroidal anti-inflammatory drugs. Addition of pyridine derivatives to non-steroidal anti-inflammatory therapy will permit to mitigate and/or prevent the decrease of prostaglandin e2 levels below physiological levels, which as a result can increase the safety and efficacy of non-steroidal anti-inflammatory drugs in said medical conditions.

The method and compositions described herein can be used in subjects in need of non-steroidal anti-inflammatory treatment to elevate the iatrogenically reduced prostaglandin e2 levels by non-steroidal anti-inflammatory treatment, and in particular in disease conditions such as disorders of the kidneys, diabetes, dysmenorrhea, but not limited to. The method and compositions described herein can thus be used to elevate reduced prostaglandin e2 levels in the tissues of a subject exposed to non-steroidal anti-inflammatory treatment in order to prevent prostaglandin e2 deficiency in said medical conditions.

The pharmacologically acceptable pyridine derivatives contemplated for use in the present embodiment are those which are chemical modifications of pyridine and having the same ring nucleus, for example nicotine, nicotinic acid and its derivatives, nicotinyl alcohol and its derivatives, pyridinol carbamate, pyridoxine and its derivatives and pyridostigmine bromide and its derivatives, but not limited to.

The pharmacologically acceptable nicotinic acid derivatives contemplated for use in the present embodiment are those which are chemical modifications of nicotinic acid, sometimes formed in the body as metabolites thereof, and having the same ring nucleus, for example nicotinamide and niceritrol, but not limited to.

The pharmacologically acceptable nicotinyl alcohol derivatives contemplated for use in the present embodiment are those which are chemical modifications of nicotinyl alcohol, and having the same ring nucleus, for example nicotinyl alcohol tartrate, but not limited to.

The pharmacologically acceptable pyridoxine derivatives contemplated for use in the present embodiment are those which are chemical modifications of pyridoxine, sometimes formed in the body as metabolites thereof, and having the same ring nucleus, for example pyridoxine, pyridoxine hydrochloride, pyridoxine-5-phosphate, pyridoxal, pyridoxal-5-phosphate, pyridoxamine, pyridoxamine-5-phosphate, 4-pyridoxic acid, but not limited to.

We contemplate that a pharmacologically acceptable pyridine derivative used in the present embodiment is nicotinic acid in any of its pharmaceutically acceptable forms, but other pharmacologically acceptable pyridine derivatives are also suitable. The amount of nicotinic acid compound or a related compound acting as a nicotinic acid (or derivative) contemplated for using in the present embodiment is from about 10 mg to about 6000 mg for administration on a daily or twice or more daily basis to an adult human patient.

The range of non-steroidal anti-inflammatory drugs with which pyridine derivatives can be beneficially included in the compositions according to the present embodiment is very wide, and extends to substantially all of the known non-selective cyclooxygenase inhibitors or/and cyclooxygenase-2 selective inhibitors currently available on the market. Specific non-steroidal anti-inflammatory drugs with which pyridine derivatives may be used in the present embodiment include naproxen, ibuprofen, diclofenac, sulindac, alclofenac, amfenac, piroxicam, fenoprofen, indomethacin, ketoprofen, flurbiprofen, alminoprofen, ketorolac, GOBAB (3-amino-4-hydroxybutyric acid), amixetrine, diflunisal, mefenamic acid, phenylbutazone, tiaprofenic acid, tolmetin and celecoxib, the various anti-inflammatory acetylsalicylates (e.g. acetylsalicylic acid) and salicylates, but not limited to.

The amount of a non-steroidal anti-inflammatory drug administered to the patient in the invented pyridine derivative use, does not normally change from the prescribed dosage being used to treat the inflammatory and other disorders in the absence of a pyridine derivative. Suitable prescribed doses vary widely according to the chosen non-steroidal anti-inflammatory drug, frequency of administration, medical conditions, clinical presentations and/or their severity and factors concerning the individual patient. Generally appropriate dosage ranges may be found by consulting standard reference pharmacopeias, and thus are well within the skill of the art. As examples, naproxen is commonly prescribed for symptoms of dysmenorrhea at an oral dosage of 500-1000 mg per day. Ibuprofen is commonly prescribed for dysmenorrhea, at a daily oral dosage of 200-1200 mg, in four separate doses per day. Acetylsalicylates are generally administered in dosages, such as 325 mg, four times per day as necessary to alleviate the symptoms. The same non-steroidal anti-inflammatory drug dosage rates as prescribed are continued in the formulations of the embodiment.

Whilst it is most convenient to prepare and use compositions which comprise a combination of a non-steroidal anti-inflammatory drug and a pyridine derivative, e.g. in a tablet or capsule form, along with suitable pharmaceutical carriers, diluents, excipients and the like, for oral administration, other methods of administration are within the scope of the present embodiment. For example, the active ingredients, namely the non-steroidal anti-inflammatory drug and the pharmacologically acceptable pyridine derivative may be administered to the patient, and the combined administration may be parenterally, intramuscularly, rectally, transcutaneously or nasally, but not limited to. Formulations of the compositions of the present embodiment for such forms of administration are standard and within the skill of the pharmaceutical compounders art.

An embodiment of the present application is a dosage form pharmaceutical composition for administration to patients requiring non-steroidal anti-inflammatory drug treatment, comprising in combination of an effective amount of a non-steroidal anti-inflammatory drug with an amount of a pharmacologically acceptable pyridine derivative effective to mitigate the prostaglandin e2 suppression side effects of the non-steroidal anti-inflammatory drug. Such a formulation suitably takes the form of an orally administrable tablet or capsule, with appropriate inert, tablet forming ingredients. The amount of non-steroidal anti-inflammatory drug in such a tablet or capsule may be in the range of 25-1000 mg, but not limited to, depending on choice of non-steroidal anti-inflammatory drug, frequency of administration, medical conditions, clinical presentations and/or their severity and factors concerning the individual patient. The amount of the pharmacologically acceptable pyridine derivative in such tablet or capsule may be in the range of 2-6000 mg, but not limited to, and depends on pharmacologically acceptable therapeutic dose of the chosen pyridine derivative.

Set forth below, by way of example and not of limitation, is a pharmaceutical composition utilizing nicotinic acid and naproxen for systemic use. Other pyridine derivatives or non-steroidal anti-inflammatory drugs of the embodiment or combination thereof, may be used in place of (or in addition to) nicotinic acid or non-steroidal anti-inflammatory drugs. The concentration of active ingredient may be varied over a wide range as discussed herein. The amounts and types of other ingredients that may be included are well known in the art.

### EXAMPLE

### Mitigation of Prostaglandin e2 Supressing Side Effect of Naproxen in Dysmenorrhea Through the Use of Nicotinic Acid

To treat or alleviate pain caused by dysmenorrhea a female patient in need of taking a daily dosage of, say, 500 mg of naproxen, takes via the oral route in one combined tablet the same prescribed 500 mg thereof together with 500 mg of nicotinic acid for mitigation of suppression of renal prostaglandin e2 levels as a side effect of naproxen.

We claim:

1. A method for mitigating prostaglandin e2 supressing side effects of non-steroidal anti-inflammatory drugs comprising administering to a mammalian patient being treated with said non-steroidal anti-inflammatory drugs a pyridine derivative to reduce prostaglandin e2 supressing side effects induced by said non-steroidal anti-inflammatory drugs.

2. The method of claim 1 wherein said pyridine derivative is selected from the group of pyridine derivatives comprising pharmaceutically acceptable forms of nicotine, nicotinic acid, nicotinamide, niceritrol, nicotinyl alcohol, nicotinyl alcohol tartrate, pyridinol carbamate, pyridostigmine bromide, pyridoxine, pyridoxine-5-phosphate, pyridoxal, pyridoxal-5-phosphate, pyridoxamine, pyridoxamine-5-phosphate and 4-pyridoxic acid or any other pharmaceutically acceptable pyridine derivatives.

3. The method of claim 1 wherein said non-steroidal anti-inflammatory drug is selected from the group of non-steroidal anti-inflammatory drugs comprising pharmaceutically acceptable forms of cyclooxygenase inhibitors and cyclooxygenase-2 inhibitors.

4. The method of claim 1 wherein said non-steroidal anti-inflammatory drug is selected from the group of non-steroidal anti-inflammatory drugs comprising salicylates, arylalkanoic acids, 2-arylpropionic acids, n-arylanthranilic acids, meloxicam, piroxicam, celecoxib, valdecoxib, lumiracoxib, etoricoxib and rofecoxib, and sulphonanilides, indomethacin, sulindac, acetylsalicylic acid, flurbiprofen, ibuprofen, naproxen drugs, and derivatives thereof.

5. A pharmaceutical preparation for mitigating prostaglandin e2 supressing side effects of non-steroidal anti-inflammatory drugs in a mammalian patient comprising a non-steroidal anti-inflammatory drug and a pyridine derivative to reduce prostaglandin e2 supressing side effects induced by said non-steroidal anti-inflammatory drug.

6. Use of pyridine derivative for the preparation of a medicament for use in combination with a non-steroidal anti-inflammatory drug for the mitigating prostaglandin e2 supressing side effects of said non-steroidal anti-inflammatory drug in a mammalian patient.
